(19) United States
(12) Patent Application Publication
(10) Pub. No.: US 2008/0176760 A1
(43) Pub. Date: Jul. 24, 2008

(54) PEPTIDE TEMPLATES FOR NANOPARTICLE SYNTHESIS OBTAINED THROUGH PCR-DRIVEN PHAGE DISPLAY METHOD

(76) Inventors: Rajesh R. Naik, Dayton, OH (US); Morley O. Stone, Bellbrook, OH (US); Daniel C. Carter, Huntsville, AL (US)

Correspondence Address: STITES & HARBISON PLLC, 1199 NORTH FAIRFAX STREET, SUITE 900, ALEXANDRIA, VA, 22314, US

(21) Appl. No.: 12/056,857

(22) Filed: Mar. 27, 2008

Related U.S. Application Data
(63) Continuation of application No. 11/045,488, filed on Jan. 31, 2005.

(51) Int. Cl. C40B 30/04; C07K 16/00; C07H 21/04

(52) U.S. Cl. 506/9; 530/300; 536/22.1

(57) ABSTRACT

A method is provided for identifying and isolating peptides capable of binding of inorganic materials such as silica, silver, germanium, cobalt, iron, or oxides thereof, or other materials on a nanometric scale such as carbon nanotubes, using a combinatorial phage display peptide library and a polymerase-chain reaction (PCR) step to obtain specific amino acids sequences. In the method of the invention, a combinatorial phage display library is used to isolate and select the desired binding peptides by a series of steps of target binding of phage with the nanometric material of interest, elution and purification of the bound phages, and amplification using PCR to determine the sequences of phages producing the desired binding peptides. The binding peptides of the invention are particularly advantageous in that they may be used as templates to guide the development of useful structures on a nanometric scale.

### CROSS-REFERENCE TO RELATED APPLICATION

This application is a continuation of U.S. Ser. No. 11/045,488, filed Jan. 31, 2005.

### FIELD OF THE INVENTION

This invention relates in general to a peptides that can bind to inorganic surfaces (metals, metal oxides and semiconductors) and other nanometric structures such as carbon nanotubes and that can thus be used to serve as templates to control the growth and nucleation of inorganic nanoparticles in vitro. These peptides include ones that can bind to diverse materials that range from metals such as silver, gold, platinum and cobalt nanoparticles, inorganic metal oxides such as silica, cobalt oxide, iron oxide, zinc oxide and tin oxide, semiconductor materials such as palladium, gadolinium, and germania to other materials such as ruby, carbon nanotubes and sodium montmorillonite, and they are preferably obtained from a phage display library using a polymerase chain reaction (PCR)-driven method. The peptides of the invention are also capable of functioning as templates for the synthesis of silver and cobalt platinum nanoparticles.

### BACKGROUND OF THE INVENTION

Biomineralization is a widespread phenomenon in nature wherein many biological systems are capable of forming structures from varied inorganic substrates. Biomolecules which have the properties of binding inorganic structures such as metals and metal oxides will be particularly important in applications on the nanometric scale. In addition, biomolecules will can extremely useful in the future because they can act as templates whereby due to their particularly properties, and thus they can be used to arrange and organize inorganic substances from the nanoparticle level and up. For example, silver, magnetite, and cadmium sulfide particles can be microbially produced, and marine organisms such as diatoms and sponges are known to synthesize siliceous structures. In general, the transformation of inorganic molecules into nano- and microstrutured components on the biological scale appears to be controlled by proteins.

Nanoparticles and nanomaterials are highly desirable in many applications because these materials have unique optical, electronic and magnetic properties that arise due to their quantum size confinement. As indicated above, there is an emerging field of nanobiotechnology which seeks to employ the use of biomolecules as templates for the synthesis of nanomaterials. Natural biological systems, which master ambient conditions chemistry, often can synthesize inorganic materials that are hierarchically organized from the nano- to the macro-scale. Numerous microorganisms are capable of synthesizing inorganic-based structures, for example, microorganisms can synthesize iron oxide, silica, silver, gold and cadmium sulfide nanoparticles.

However, the use of proteins and other biomaterials from such microorganisms to possibly direct the assembly of nanostructured components into sophisticated functional structures has remained hard if not impossible to accomplish efficiently and has long been a desired goal. The ability to utilize proteins to produce nanostructured inorganic materials, such as those made of silica or other similar compounds in vivo under ambient conditions would provide a significant advantage over traditional approaches to materials synthesis which require stringent conditions such as high nanometric metallic or other inorganic materials using a biomimetic approach which will only require ambient conditions to produce useful inorganic structures such as those useful on a nanometric scale with a minimum amount of complexity and expense.

In the biological arts, it has long been known to utilize a phage display library to express a particular protein. Phage peptide display is a selection technique in which random peptides from a library are expressed as a fusion with a phage coat protein, resulting in the display of the fused protein on the surface of the phage particle. The advantage of phage display technology is that it can offer the ability to identify surface-specific proteins in a more practical way and avoid the lengthy and complex identification procedures associated with traditional protein isolation and gene sequencing. However, it has not previously been known to utilize phage display technology in such a manner as to identify and produce peptides which can exhibit binding and nucleation properties against an inorganic material such as silica, silver, cobalt, iron, etc., in order to direct the precipitation of these materials so as to be able to create useful structures on a nanometer scale. Accordingly, it is thus highly desirable to develop a method for utilizing phage display libraries in order to allow rapid selection of surface-specific peptides and identify a subpopulation of silica-precipitating peptides, or peptides that can be used to catalyze the precipitation or deposition of other inorganic materials, and to use such peptides as templates for “bottom-up” microfabrication.

In addition, there are numerous applications wherein the delivery or removal of inorganic agents plays an important role in the complexity, expense and efficiency of the particular method. For example, in the case of toxic waste areas, it is very often the case that the most toxic ingredients that need to be removed from a site are the heavy metals which are extremely toxic and sometimes even radioactive. In the case of the infamous Love Canal site, metals in toxic levels discovered at the site included aluminum, antimony, beryllium, cadmium, copper, iron, lead, selenium, silver and zinc. At present, although there are many known methods for attempting to remove toxic levels of metals at such a site, many of these methods are often expensive, inefficient and general in nature and thus may not be adequate to eliminate or reduce levels of particular metals. Even further, it is important to be able to clean up and contain desirable to achieve a method and product whereby such radioactive materials can be specifically bound and removed when necessary.

The use of phage peptide display libraries to select peptides that bind to inorganic surfaces has been disclosed, for example, in PCT Published Application WO 03/078,451, incorporated herein by reference. However, this system involved the labor-intensive procedures of phage amplification which would result in slowing down the process and in making it more expensive and cumbersome.

It is thus still remains desirable to utilize these methods and develop peptides which can be used as templates in order to promote synthesis of inorganic or organic materials on a nanometric scale, but with improved efficiency so that it can be conducted on a larger yet less expensive scale.

### SUMMARY OF THE INVENTION

Accordingly, it is thus an object of the present invention to provide peptides that can bind to inorganic surfaces (metals, metal oxides and semiconductors) or other objects (e.g., carbon nanotubes) on a nanometric scale and which can thus be used to serve as templates to control the growth and nucleation of inorganic nanoparticles in vitro.

It is further an object of the present invention to provide a system wherein a phage display peptide library is used in the identification and isolation of peptides which can bind silica or other nanometric materials and which can thus be useful as templates in methods of fabricating structures on an nanometric scale.

It is still further an object of the present invention to isolate and identify useful peptides which can bind to inorganic or organic materials using a method that is quick, efficient, and which can be carried out with a minimum of steps and without the need for rigorous physical conditions.

It is still further an object of the present invention to develop and provide peptides which can be used to catalyze the precipitation and deposition of useful inorganic materials such as silica, silver, germanium, cobalt oxide, iron oxide and other metals and metal oxides.

It is yet a further object of the present invention to develop and provide peptides which can be used as templates to direct the development of many functional nanometric materials such as carbon nanotubes.

It is even further an object of the present invention to develop and provide peptides which can be used as templates for the synthesis of nanoparticles from inorganic materials such as silver, gold, platinum, cobalt, silica, iron, zinc, tin, palladium, gadolinium, germanium, and oxides thereof.

It is yet further an object of the present invention to isolate and identify useful peptides which can bind to potentially toxic inorganic materials and thus be used in methods of delivering or removing said materials when necessary.

It is yet further an object of the present invention to develop and provide peptides which can be used to remove or delivery radioactive materials in an efficient and relatively inexpensive manner.

These and other objects are achieved by virtue of the present invention which provides a method for identifying and isolating peptides capable of binding to inorganic materials and other nanometric particles such as carbon nanotubes using a combinatorial phage display peptide library and a step involving the polymerase-chain reaction (PCR). By the present method, it is possible to eliminate the labor-intensive and inefficient procedures of phage amplification as used in prior art methods and directly obtain sequence information of interacting peptides in a single step using the PCR method. In the method in accordance with the invention, a combinatorial phage display library is used to isolate and select the desired binding peptides by a series of steps of target binding, elution and amplification which may be repeated until the desired amount of phage expressing peptides with the desired binding properties is obtained. Once these phage are isolated and/or purified following this procedure, the phage coating is ruptured or otherwise removed so as to release the phage nucleic acids, and a step involving the polymerase-chain reaction is utilized in order to obtain the sequences of the peptides binding to the particular nanoparticles introduced to the phage display library. Once identified in this manner, the peptides then may be expressed and used as templates to guide the precipitation and synthesis of useful structures on a nanometric scale.

These and other features of the present invention as set forth in, or will become obvious from, the detailed description of the preferred embodiments provided hereinbelow.

### BRIEF DESCRIPTION OF THE DRAWING FIGURES

FIG. 1 is a schematic depiction of a process in accordance with the present invention.

FIG. 2 shows peptide-displaying phages remain bound to Ag nanoparticles after acid elution. Agarose gel electrophoresis of PCR-amplified peptide-displaying phage DNA observed in acid eluted Ag nanoparticles (lane 1) but not in acid eluted ZnS nanoparticles (lane 2). The positive control amplified DNA product obtained using 50 non-specific peptide-displaying phages (lane 3). The absence of an amplified DNA fragment when one DNA primer is used in the PCR reaction (lane 4).

FIG. 3 shows the synthesis of silver nanoparticles. (A) Incubation of peptides with 0.2 mM silver nitrate on the laboratory bench top for 24-48 hr resulted in the formation of a yellowish-red colored solution. Silver nitrate solution lacking peptide was colorless. (B) TEM analysis of AG-P35 synthesized nanoparticles. (C) UV-Vis spectrum of the solutions shown in panel A.

FIG. 4 shows peptide-displaying phages bind to uneluted and acid eluted cobalt nanoparticles. DNA gel electrophoresis showing the intensity of the peptide-displaying phage DNA fragment obtained from the uneluted sample (lane 2) is greater than from the eluted samples (lane 3). Amplified DNA from 50 peptide-display phages were run in parallel for comparison (lane 4) and no amplified DNA fragment was visible in the absence of phage DNA. Lane 1 shows the migration of DNA molecular weight markers on the agarose gel.

FIG. 5 shows a Venn diagram comparing results from methods including the method in accordance with the present invention.

FIG. 6 shows the Synthesis of CoPt nanoparticles. (A) Solutions of CoPt nanoparticles, dispersion of the Co1-P10 peptide stabilized CoPt nanoparticles, arrow indicates the accumulated precipitate at the bottom of the glass vials lacking peptide or in the presence of Co1-P15 peptide. (B) TEM micrograph of Co1-P10 synthesized CoPt nanoparticles. (C) TEM image of individual nanoparticles and (D) HRTEM of a single CoPt nanoparticle showing the lattice fringes.

FIG. 7 shows inorganic binding peptides that can be obtained in accordance with the present invention.

FIG. 8 shows additional inorganic binding peptides that can be obtained in accordance with the present invention.

### DETAILED DESCRIPTION OF THE PREFERRED EMBODIMENTS

In accordance with the present invention, there is provided a method for utilizing a combinatorial phage display library to identify and obtain peptides which can bind to nanometric particles including organic materials such as carbon nanotubes and inorganic materials such as silica and other metals and metal oxides and which can be used to catalyze the precipitation and deposition of those materials on a nano/micrometer scale. In the preferred process, the nanometric material-binding peptides are obtained using a suitable combinatorial phage display peptide library such as would be commercially available and well known in the art. Phage peptide display is a selection technique in which a library of random peptides are expressed as a fusion with a phage coat protein resulting in the display of the fused protein on the surface of the phage particle. In the present invention, a suitable combinatorial library will be one in which phage expressing peptides binding to metals, metal oxides, and other inorganic and organic materials on the nanometric scale can be identified. One such combinatorial phage display library is the 12 amino acid phage peptide display library (PhD-12) was purchased from New England Biolabs, Inc (Beverly, Mass.). The phage-display peptide library consists of 10⁹ different phage clones, each displaying a unique 12 amino acid peptide on the phage surface.

In the general process of the invention, as explained further below, the nanometric material-binding peptides of the invention are obtained by first incubating the target particles with the combinatorial phage display peptide library. As indicated below, target particles in accordance with the invention will include any suitable inorganic or organic material (e.g., carbon nanotubes) that can be bound by peptides and which can be deposited or precipitated to form an appropriate nanostructure. Examples of such inorganic materials include metals and metal oxides currently used in applications on a nanometric scale including silver, gold, platinum, cobalt, silica, iron, zinc, tin, palladium, gadolinium, germanium, and oxides thereof. In addition, it is also possible to bind other inorganic materials which may be important in methods of removing or delivering these metals or oxides for various purposes. Accordingly, inorganic materials that can be removed using the process of the invention include aluminum, antimony, beryllium, cadmium, copper, iron, lead, selenium, silver and zinc. Still other materials such as those which would be potentially recoverable from aqueous environments such as lakes, streams, etc., and these would include gold, platinum, palladium, and oxides thereof. Further, it is also desired to develop means of either removing or delivering radioactive metals, such as uranium, radioactive cobalt, etc., and thus these metals as well can be bound using the peptides prepared in accordance with the process of the invention, as explained further below. In short, the diverse nanometric materials that can be utilized in conjunction with the present method range from metals such as silver, gold, platinum and cobalt nanoparticles, inorganic metal oxides such as silica, cobalt oxide, iron oxide, zinc oxide and tin oxide, semiconductor materials such as palladium, gadolinium, and germania to other materials such as ruby, carbon nanotubes and Na⁺ montmorillonite.

The present invention thus can be used to obtain peptides that will specifically bind with all of the inorganic elements conventionally known, where stable in the pure form, such as would be reflected in the Periodic table of elements. It is also contemplated that the present method will be useful to isolate and/or identify peptides which can bind to these inorganic elements along with their stable inorganic complexes as well, e.g., oxides, etc. of these elements.

In the preferred process, as explained further below, the phages from the combinatorial library are identified and sequenced using a PCR process and selected for their ability to express peptides that exhibit selective affinity for a particular nanometric material and which will be able to guide the deposition and precipitation of that material such as in the form of a template for nanometric structures. In the preferred process in accordance with the invention, peptides which can bind to an inorganic material or other nanometric materials such as carbon nanotubes can be isolated and identified via the steps of incubating a combinatorial phage peptide display peptides expressed by the phage of the library; eluting the library so as to collect the phage bound to the target material; rupturing the phage so as to release the nucleic acid of the phage bound to the target material; amplifying the nucleic acid of the phage bound to the target material using a polymerase-chain reaction (PCR); and sequencing the phage nucleic acid so as to determine the sequence of the peptides coded by the nucleic acid of the phage which can bind to the target nanometric material.

As indicated above, in accordance with the present invention, it is preferred that a combinatorial phage display peptide library such as the 12 amino acid phage peptide display library (PhD-12) be utilized in the invention, however, other available amino acid libraries having suitable peptides of other lengths would also be useful in the invention. In the preferred process, the phage display library is incubated with the desired target material as described above so as to target phage which express peptides capable of binding with that material. Accordingly, the invention includes a step of isolating the desired phage such as by eluting the phage bound to the target and separating and collecting the desired phage. The use of the target particle to identify and isolate phage which express the peptides with the desired binding properties is known as “panning” or “biopanning”, and in the preferred process, multiple rounds of panning may be carried out as desired to further purify the selected phage and increase the likelihood that the eluted and isolated phage will bind specifically to the target material. Ideally, the target particle is first itself isolated and purified before being used in the present process, such as through an acid wash or other suitable purification process.

In the preferred process of the invention, the method provides steps which go beyond the “panning” process and allow for the identification and isolation of many more binding peptides than was possible using previous methods. In this process wherein peptides are isolated via incubation of a phage peptide display library with a target nanometric material for a time sufficient to allow binding of the material to the peptides expressed by the phage of the library, the incubation step may be conducted using an appropriate phage display library (e.g., the Ph.D.12 library described above) and the desired nanometric material in a medium (e.g., a Tris-buffered saline solution containing 0.5% Tween 20, or TBST) for a time (e.g., 1 hour) and at a temperature (e.g., room temperature) suitable to allow the target material to bind with the peptides of the phage. This incubation step may optionally be followed by a step of washing the phages with a suitable buffer solution in order to assist in eliminating phages that do not specifically bind to the target material. Following the wash step, the phages of the library are eluted to collect the phage bound to the target material via a suitable elution step, e.g., through use of a suitable elutant such as 0.2 M glycine-HCl (pH 2.2) for a suitable time (e.g., 20 minutes).

The next step of the preferred process is to rupture the phage so as to release the nucleic acid of the phage bound to the target material, and this can be done in a number of suitable ways well known in the art. For example, the bound phage can be treated with a lysis solution (such as lysis buffer A comprised of 10 mM Tris-HCl pH 8.3, 10 mM ethylenediaminetetraacetic acid (EDTA), and 1% Triton X-100) for a time and at a temperature (e.g., 10 minutes at 95° C.) suitable to allow rupture of the phage coat and release of the phage DNA. At this point, the nucleic acid of the phage binding peptides can be sequenced using the techniques described, for example, in PCT application WO 01/79479, incorporated herein by reference. In the preferred process, once the desired phage expressing the peptides are isolated and/or purified as described above, the nucleic acid (e.g., DNA) from the selected phages are then isolated and sequenced to obtain the genetic information encoding for the displayed peptides and determine the sequence of the peptides coded by the nucleic acid of the phage which can bind to the target nanometric material. This may occur through a number of suitable techniques well known in the art including amplification of the genetic material by known processes such as an automated sequencer or other suitable PCR techniques.

In the particularly preferred process, such as those recommended by the manufacturer, a suitable amount of the PCR materials (e.g., 50 μl of PCR REDTaq ReadyMix PCR master mix manufactured by Sigma-Aldrich, St. Louis, Mo.) was directly added to the tube containing the lysed peptide displaying phages. The phage DNA sequences in this process can be amplified such as by adding to the tube 1 μl each of forward (5′-CCTCGAAAGCAAGCTGATAAC-3′) and reverse (5′-GTACCGTMCACTGAGTTTCG-3′) primers. The PCR amplification can be performed for a suitable time, e.g., using 20-25 cycles of denaturation at 95° C. for 30 sec, extension cycle of 72° C. for 5 min. The PCR products can then be separated such as on a 1.2% agarose gel by DNA electrophoresis. The PCR products are then preferably cloned into the TOPO vector (Invitrogen, San Diego, Calif.) according to manufacturer's instructions. The clones obtained in the present invention can then be sequenced using an automated DNA sequencer using standard sequencing methods.

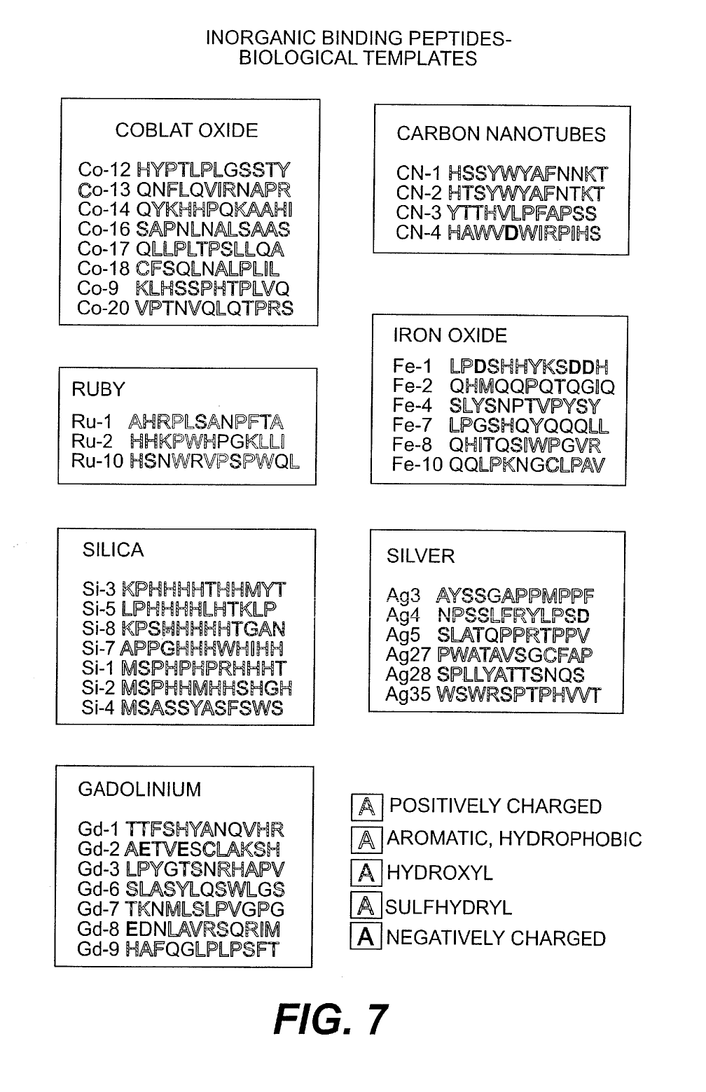

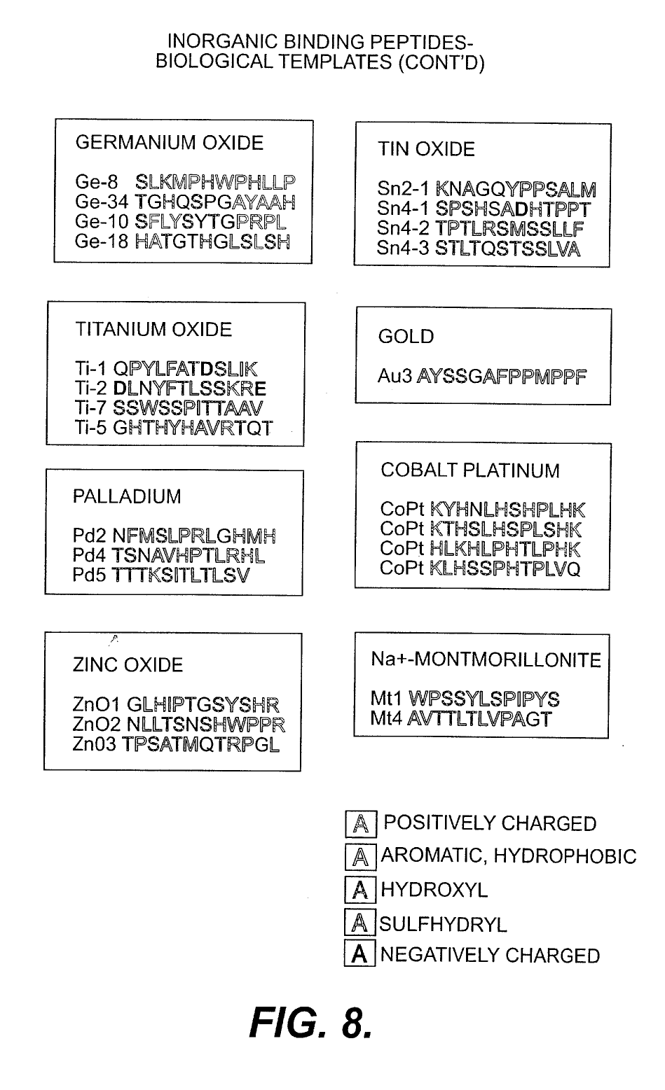

As indicated above, the process of the invention can be carried out on any desired nanometric material, such as an inorganic metal or metal oxide or other useful nanometric structure such as carbon nanotubes, that may be used in applications where such materials may be deposited or precipitated on a nanometric scale to form suitable structures such as those used in sensor arrays, microchips, etc. Among the inorganic materials useful in the invention are silica, silver, germanium, cobalt, iron, and the oxides of these metals and those other inorganic materials described above. Accordingly, the present invention includes the production or isolation and identification of those phage expressing peptides which bind to the target nanometric materials as well as to the amino acid sequences of the expressed peptides and the nucleic acid sequences encoding said amino acids. Examples of suitable peptides that can be obtained in accordance with the invention are those shown in FIGS. 7 and 8, and which are summarized herein as follows:

Silica Binding Peptides:

| | | |
|---|---|---|
| Si-3 | KPHHHHTHHMYT | (SEQ ID NO: 1) |
| Si-5 | LPHHHHLHTKLP | (SEQ ID NO: 2) |
| Si-8 | KPSHHHHHTGAN | (SEQ ID NO: 3) |
| Si-7 | APGHHHWHIHH | (SEQ ID NO: 4) |
| Si-1 | MSPHPHPRHHHT | (SEQ ID NO: 5) |
| Si-2 | MSPHHMHHSHGH | (SEQ ID NO: 6) |
| Si-4 | MSASSYASFSWS | (SEQ ID NO: 7) |

Silver Binding Peptides:

| | | |
|---|---|---|
| AG-P1 | KFLQFVCLGVGP | (SEQ ID NO: 8) |
| AG-P2 | AVLMQKYHQLGP | (SEQ ID NO: 9) |
| AG-P3 | IRPAIHIIPISH | (SEQ ID NO: 10) |
| AG-P4 | NVIRASPPDTSY | (SEQ ID NO: 11) |
| AG-P5 | LAMPNTQADAPF | (SEQ ID NO: 12) |
| AG-P6 | QQNVPASGTCSI | (SEQ ID NO: 13) |
| AG-P10 | NAMPGMVAWLCR | (SEQ ID NO: 14) |
| AG-P11 | HNTSPSPIILTP | (SEQ ID NO: 15) |
| AG-P12 | ASQTLLLPVPPL | (SEQ ID NO: 16) |
| AG-P14 | YNKDRYEMQAPP | (SEQ ID NO: 17) |
| AG-P18 | TLLLLAFVHTRH | (SEQ ID NO: 18) |
| AG-P27 | PWATAVSGCFAP | (SEQ ID NO: 19) |
| AG-P28 | SPLYATTSNQS | (SEQ ID NO: 20) |
| AG-P35 | WSWRSPTPHVVT | (SEQ ID NO: 21) |
| Ag3 | AYSSGAPPMPPF | (SEQ ID NO: 22) |
| Ag4 | NPSSLFRYLPSD | (SEQ ID NO: 23) |
| Ag5 | SLATQPPRTPPV | (SEQ ID NO: 24) |

Cobalt oxide binding peptides

| | | |
|---|---|---|
| Co1-P6 | GVLNAAQTWALS | (SEQ ID NO: 25) |
| Co1-P10 | HYPTLPLGSSTY | (SEQ ID NO: 26) |
| Co1-P15 | QYKHHPQKAAHI | (SEQ ID NO: 27) |
| Co1-P17 | HPPTDGMVPSPP | (SEQ ID NO: 28) |
| Co2-P9 | TQQTDSRPPVLL | (SEQ ID NO: 29) |
| Co2-P11 | TFPSHLATSTQP | (SEQ ID NO: 30) |
| Co2-P17 | QLLPLTPSLLQA | (SEQ ID NO: 31) |
| Co3-p16 | VPTNVQLQTPRS | (SEQ ID NO: 32) |
| Co-18 | CFSQLNALPLIL | (SEQ ID NO: 33) |
| Co1-P1 | HSVRWLLPGAHP | (SEQ ID NO: 34) |
| Co1-P2 | HETNPPATIMPH | (SEQ ID NO: 35) |
| Co1-P3 | WASAAWLVHSTI | (SEQ ID NO: 36) |
| Co1-P4 | SPLQVLPYQGYV | (SEQ ID NO: 37) |
| Co1-P5 | ESIPALAGLSDK | (SEQ ID NO: 38) |
| Co1-P8 | TPNSDALLTPAL | (SEQ ID NO: 39) |
| Co1-P13 | HAMRPQVHPNYA | (SEQ ID NO: 40) |
| Co1-P16 | YGNQTPYWYPHR | (SEQ ID NO: 41) |
| Co1-P18 | TWQPFGMRPSDP | (SEQ ID NO: 42) |
| Co1-P21 | TGDVSNNPNVTL | (SEQ ID NO: 43) |
| Co2-P3 | SAPNLNALSAAS | (SEQ ID NO: 44) |
| Co2-P5 | SVSVGMKPSPRP | (SEQ ID NO: 45) |
| Co2-P1 | SLTQTVTPWAFY | (SEQ ID NO: 46) |
| Co2-P7 | TNLDPSYPLHHL | (SEQ ID NO: 47) |
| Co2-P13 | QNFLQVIRNAPR | (SEQ ID NO: 48) |
| Co2-P2 | KLHSSPHTPLVQ | (SEQ ID NO: 49) |
| Co3-P12 | GTSTFNSVPVRD | (SEQ ID NO: 50) |

Iron Oxide binding peptides

| | | |
|---|---|---|
| Fe-1 | LPDSHHYKSDDH | (SEQ ID NO: 51) |
| Fe-2 | QHMQQPQTQGIQ | (SEQ ID NO: 52) |
| Fe-4 | SLYSNPTVPYSY | (SEQ ID NO: 53) |
| Fe-7 | LPGSHQYQQQLL | (SEQ ID NO: 54) |
| Fe-8 | QHITQSIWPGVR | (SEQ ID NO: 55) |
| Fe-10 | QQLPKNGCLPAV | (SEQ ID NO: 56) |

Germanium Oxide binding peptides

| | | |
|---|---|---|
| Ge-8 | SLKMPHWPHLLP | (SEQ ID NO: 57) |
| Ge-34 | TGHQSPGAYAAH | (SEQ ID NO: 58) |
| Ge-10 | SFLYSYTGPRPL | (SEQ ID NO: 59) |
| Ge-18 | HATGTHGLSLSH | (SEQ ID NO: 60) |

Tin Oxide binding peptides

| | | |
|---|---|---|
| Sn2-1 | KNAGQYPPSALM | (SEQ ID NO: 61) |
| Sn4-1 | SPSHSADHTPPT | (SEQ ID NO: 62) |
| Sn4-2 | TPTLRSMSSLLF | (SEQ ID NO: 63) |
| Sn4-3 | STLTQSTSSLVA | (SEQ ID NO: 64) |

Titanium Oxide binding peptides

| | | |
|---|---|---|
| Ti-1 | QPYLFATDSLIK | (SEQ ID NO: 65) |
| Ti-2 | DLNYFTLSSKRE | (SEQ ID NO: 66) |
| Ti-7 | SSWSSPITTAAV | (SEQ ID NO: 67) |
| Ti-5 | GHTHYHAVRTQT | (SEQ ID NO: 68) |

Gadolinium binding peptides

| | | |
|---|---|---|
| Gd-1 | TTFSHYANQVHR | (SEQ ID NO: 69) |
| Gd-2 | AETVESCLAKSH | (SEQ ID NO: 70) |
| Gd-3 | LPYGTSNRHAPV | (SEQ ID NO: 71) |
| Gd-6 | SLASYLQSWLGS | (SEQ ID NO: 72) |
| Gd-7 | TKNMLSLPVGPG | (SEQ ID NO: 73) |
| Gd-8 | EDNLAVRSQRIM | (SEQ ID NO: 74) |
| Gd-9 | HAFQGLPLPSFT | (SEQ ID NO: 75) |

Ruby binding peptides

| | | |
|---|---|---|
| Ru-1 | AHRPLSANPFTA | (SEQ ID NO: 76) |
| Ru-2 | HHKPWHPGKLLI | (SEQ ID NO: 77) |
| Ru-10 | HSNWRVPSPWQL | (SEQ ID NO: 78) |

Carbon Nanotube binding peptides

| | | |
|---|---|---|
| CN-1 | HSSYWYAFNNKT | (SEQ ID NO: 79) |
| CN-2 | HTSYWYAFNTKT | (SEQ ID NO: 80) |
| CN-3 | YTTHVLPFAPSS | (SEQ ID NO: 81) |
| CN-4 | HAWVDWIRPIHS | (SEQ ID NO: 82) |

Cobalt Platinum binding peptides

| | | |
|---|---|---|
| CoPt | KYHNLHSHPLHK | (SEQ ID NO: 83) |
| CoPt | KTHSLHSPLSHK | (SEQ ID NO: 84) |
| CoPt | HLKHLPHTLPHK | (SEQ ID NO: 85) |
| CoPt | KLHSSPHTPLVQ | (SEQ ID NO: 86) |

Palladium binding peptides

| | | |
|---|---|---|
| Pd2 | NFMSLPRLGHMH | (SEQ ID NO: 87) |
| Pd4 | TSNAVHPTLRHL | (SEQ ID NO: 88) |
| Pd5 | TTTKSITLTLSV | (SEQ ID NO: 89) |

Zinc Oxide binding peptides

| | | |
|---|---|---|
| ZnO1 | GLHIPTGSYSHR | (SEQ ID NO: 90) |
| ZnO2 | NLLTSNSHWPPR | (SEQ ID NO: 91) |
| ZnO3 | TPSATMQTRPGL | (SEQ ID NO: 92) |

Gold binding peptides

| | | |
|---|---|---|
| Au3 | AYSSGAFPPMPPF | (SEQ ID NO: 93) |

Na⁺Montmorillonite binding peptides

| | | |
|---|---|---|
| Mt1 | WPSSYLSPIPYS | (SEQ ID NO: 94) |
| Mt4 | AVTTLTLVPAGT | (SEQ ID NO: 95) |

As indicated above, these peptides may be expressed and utilized in a number of suitable applications as would be understood by one skilled in the art, but in particular, the nanometric material-binding peptides obtained by virtue of the present invention are ideally used as templates in nanometric material synthesis and may be used to guide the development of important microstructures on a nanometric scale such as would be used in sensors, computer chips and the like. In the preferred embodiments, the peptides obtained in accordance with the invention will be suitable for catalyzing and promoting the precipitation or directing growth of the particular inorganic material which is the target of the peptide of the invention.

In addition to the embodiments described above, the present invention may be used in a number of beneficial applications. For example, it is also possible to bind other inorganic materials which may be important in methods of removing or delivering these metals or oxides for various purposes. Accordingly, the present invention contemplates a method for recovering an inorganic material using a peptide identified and/or isolated in accordance with the invention as set forth above, by introducing an amount of the said peptide to the area or site where recovery or elimination of the particular inorganic material is desired, maintaining said peptide at said site for a time effective to achieve the desired level of peptide binding to the inorganic material, and then removing the bound peptide so as to recover or eliminate the particular metal bound by the peptide. In these applications, the effective amount of the peptide will vary depending on the type of application, and one skilled in the art would appreciate that each individual job would have an appropriate amount of peptide depending on the circumstances of the application.

For example, the peptides of the invention can be used in cases wherein recovery of a valuable inorganic material is desired, such as the mining of metals such as silver, gold or platinum from lakes and streams. In these applications, a number of suitable ways could be used to carry out such removal, including use of synthesized versions of the binding peptides identified from the above process, or via a recombinant genetic vector, such as an bacterial organism using a plasmid or viral vector with the genetic instructions to express the peptides in accordance with the invention. One suitable vector would be to have the peptides expressed in E. coli which could be prepared in suitable amounts, introduced to the body of water, whether natural stream or lake or artificial enclosure such as a tank or vat, given suitable time for the expression of the peptides and the binding to the precious metals, and then recovery or filtering of the peptides bound to the precious metals, following which the peptides could be separated through various means well known in the art. In addition, it is possible to prepare the peptides or E. coli expressing such peptides in a suitable vehicle, such as a filter or cartridge, e.g., where the peptides could be linked to a solid support (e.g., agarose, resins, polysaccharides, etc.) in such a way that their binding site is unaffected. Similar steps could be taken where the inorganic material is a toxic product, e.g., toxic metal waste or radioactive waste, and in these cases, the recovery of the metal in this procedure would be followed by its disposal or containment in a suitable manner. In addition, such peptides could be used in chelation methods of eliminating targeted inorganic materials from a human or animal patient.

Similarly, the present invention contemplates applications wherein delivery of a particular inorganic material is highly desirable, such as the direct application of a radioactive material, such as radioactive cobalt, to a tumorous cell in a cancer patient. In general, the method of delivering an inorganic material bound to a peptide identified and/or isolated in accordance with the invention as set forth above, can be carried out binding the peptide to the desired inorganic agent, and linking the bound peptide to a means of delivering the bound peptide to a particular site. For example, in the case of cancer treatment, it is possible to link the peptide to an antibody that can target particular tissues, such as cancerous tissues or cells, and the antibody-peptide-bound inorganic material complex can be introduced in a patient where it will apply the necessary agent, e.g., radioactive cobalt, directly to the site of the tumor. In such a case, the peptide will be appropriately linked to the antibody in such a manner that the binding property of the peptide to the inorganic material is unaffected and the target binding site of the antibody is unaffected.

Still other applications of the peptides of the invention would be in an area or site where recovery or elimination of the particular inorganic material is desired, maintaining said peptide at said site for a time effective to achieve the desired level of peptide binding to the inorganic material, and then removing the bound peptide so as to recover or eliminate the particular metal bound by the peptide. In these applications, the effective amount of the peptide will vary depending on the type of application, and one skilled in the art would appreciate that each individual job would have an appropriate amount of peptide depending on the circumstances of the application. Such a method will be useful in applications such as recovery or valuable metals or elimination of toxic inorganic materials. Another application of specific high binding peptides in accordance with the invention is that they can be directed to metals applied in NMR and X-ray contrast agents (e.g., as carried by the fragment or fused to a larger protein).

In accordance with the invention, a method can also be utilized for obtaining phage which can express the peptides of the invention. In such methods, the phage is obtained in a method comprising the steps of incubating a combinatorial phage display peptide library with a target inorganic material which will bind to peptides expressed by the phage of the library; eluting the library so as to collect the phage bound to the target inorganic material; and amplifying the nucleic acid of the phage bound to the target inorganic material using a polymerase-chain reaction (PCR) and sequencing said nucleic acid so as to determine the sequence of the peptides coded by the nucleic acid of the phage which can bind to the target inorganic material.

In addition, the present method can be used to identify peptides which can bind to a stable inorganic element or a stable inorganic complex of that element using a combinatorial phage display library as described above. In such a method, these peptides are identified by the steps of incubating a combinatorial phage display peptide library with a target inorganic element or complex which will bind to peptides expressed by the phage of the library; eluting the library so as to collect the phage bound to the target inorganic element or complex; isolating the nucleic acid of the phage bound to the target inorganic element or complex; amplifying the nucleic acid of the phage bound to the target inorganic element or complex using a polymerase-chain reaction (PCR); and sequencing the nucleic acid so as to determine the sequence of the peptides coded by the nucleic acid of the phage which can bind to the target inorganic element or complex.

It is thus submitted that the foregoing embodiments are only illustrative of the claimed invention and not limiting of the invention in any way, and alternative embodiments that would be obvious to one skilled in the art not specifically set forth above also fall within the scope of the claims.

The following examples are presented as illustrative of the present invention or methods of carrying out the invention, and are not restrictive or limiting of the scope of the invention in any manner.

### EXAMPLES

### Example 1

### Isolation and Identification of Peptide Templates on a Nanometric Scale Using the Methods In Accordance With the Invention

### Overview

Phage peptide display libraries are commonly used to select for peptides that bind to inorganic surfaces (metals, metal oxides and semiconductors). These binding peptides can serve as templates to control the nucleation and growth of inorganic nanoparticles in vitro. In this report, we describe the identification of a unique set of sequences that bind to silver and cobalt nanoparticles from a phage peptide display library using a Polymerase Chain Reaction (PCR)-driven method. The amino acid sequences obtained by the PCR method are a distinct set of sequences that would otherwise be missed using the regular panning method. Peptides identified by the method described here are also shown to function as templates for the synthesis of silver and cobalt nanoparticles.

### Background

Nanomaterials have unique optical, electronic and magnetic properties that arise due to their quantum size confinement. Nanobiotechnology, an emerging discipline, seeks to employ the use of biomolecules as templates for the synthesis of nanomaterials. Biological systems, a master of ambient conditions chemistry, synthesize inorganic materials that are hierarchically organized from the nano- to the macro-scale. Numerous microorganisms are capable of synthesizing inorganic-based structures, for example, microorganisms can synthesize iron oxide, silica, silver, gold and cadmium sulfide nanoparticles.¹⁻⁷ The process of biomineralization and assembly of nanostructured inorganic components into hierarchical structures has led to the development of a variety of approaches that mimic the recognition and nucleation capabilities found in biomolecules for inorganic material synthesis. A number of studies have demonstrated that proteins identified from biological organisms can be used as catalysts or templates for material synthesis in vitro.⁸⁻¹¹

These proteins control the nucleation and growth of the inorganic structure. Amino acids are known to interact with metal ions, for example, histidine, arginine, lysine, cysteine, methionine, tryptophan, aspartate, and glutamate are known to form complexes with metal ions.¹²⁻¹⁵ The presence of a particular sequence of amino acids within a peptide provides the molecular recognition motif required to interact with a given target (metals, metal oxides, semiconductors). Random peptide libraries are screened to select or evolve ligands with specific amino acid sequences that recognize the inorganic surfaces.¹⁶⁻²³ This approach of “evolution in a test tube” allows for the selection of amino acid sequences that exhibit high affinity for inorganic surfaces. In some cases, the specific molecular recognition properties of selected peptides provided by the amino acid functional groups can be used to control the nucleation and growth of the material it was selected against.

Phage peptide display is a powerful technique for selecting peptides with novel properties.²⁴ In phage peptide display, a combinatorial library of random peptides (˜10⁹) is usually expressed as a fusion protein with the phage minor coat protein (pill). As a result, the peptide is displayed on the outer surface of the phage particle with each phage particle displaying 5 copies of the same 12 or 7 amino acid peptide sequence. Since the DNA sequence for the displayed peptide is genetically fused to the pill gene, recovery of the phage DNA allows for deciphering the amino acid sequence of the selected peptide. The procedure, also know as “panning”, is carried out by incubating the library of phage displayed peptides with a target, washing away unbound/non-specific phages, and eluting the bound phage using a low pH incubation step. The eluted phage are amplified and subjected to additional panning rounds to evolve highly enriched binding sequences. Finally, the DNA from individual phage clones is isolated and sequenced. We have observed that a number of phage displayed peptides remain strongly bound to the target even after an extended acid elution step. Since these strong binding sequences are essentially lost during the regular panning method, many peptides with the highest affinity for the target are potentially missed. If the goal is to use these peptides as templates for the nucleation and growth of materials that they were selected against, then a unique peptide set has been overlooked in work to date. In this report, we show the rapid identification of peptides that interact with silver and cobalt nanoparticles and demonstrate that these peptides are efficient templates in the synthesis of silver and cobalt nanoparticles.

In an earlier study, we identified three peptides from the phage peptide display library that interacted with silver nanoparticles using the regular acid elution-based panning method.²² Only one out of the three silver binding peptides was capable of efficient silver nanoparticle synthesis. This peptide, AG4, reduced silver ions to metallic silver without the need of an external reducing agent.²² The identification of only three binding sequences from the combinatorial library prompted us to confirm whether all the peptide displaying phages were eluted from the surface of the silver nanoparticles.

### Experimental Procedures

PHAGE PEPTIDE DISPLAY SCREENING: Silver and cobalt binding peptides were selected using the Ph.D.-12 phage display peptide library obtained from New England Biolabs, Inc (Beverly, Mass.). The target binding, elution and amplification were carried out according to manufacturer's instructions. Briefly, the peptide library was incubated with acid washed silver or cobalt nanoparticles (Sigma-Aldrich, St Louis, Mo.) in Tris-buffered saline containing 0.5% Tween-20 (TBST) for 1 hr at room temperature, followed by several washes in TBST buffer (0.5%-0.8% Tween 20). The phages were eluted from the particles by the addition of 0.2 M glycine-HCl (pH 2.2) for 20 minutes. The eluted phage were then transferred to a fresh tube and neutralized with Tris-HCl, pH 9.1. The eluted phage were then tittered and subjected to 3 additional pannings (Set I). After the final panning procedure, Esherichia coli ER2537 host cells were infected with the eluted phage and plated on Luria Broth (LB) plates containing X-Gal and IPTG. DNA was isolated from 30 independent blue plaques and sequenced using an ABI 310 (PE Applied Biosystems, CA) automated sequencer.
PCR METHOD: The PCR method was performed as described with some minor modifications²⁵. Binding of the phage peptide library with the nanoparticles was done as described above. The phages were either washed several times in TBST buffer only (Set II) or washed in TBST buffer followed by elution with 0.2 M glycine-HCl pH 2.2 (Set I). The phage-nanoparticle complex was incubated in 48 μl lysis buffer A (10 mM Tris-HCl pH 8.3, 10 mM EDTA, 1% Triton X-100) for 10 min at 95° C. 50 μl PCR REDTaq ReadyMix PCR master mix (Sigma-Aldrich, St. Louis, Mo.) was directly added to the tube containing the lysed peptide displaying phages. The phage DNA sequences were amplified by adding to the tube 1 μl each of forward (5′-CCTCGAAAGCAAGCTGATAAC-3′) and reverse (5′-GTACCGTMCACTGAGTTTCG-3′) primers. The PCR amplification was performed using 20-25 cycles of denaturation at 95° C. for 30 sec, annealing at 50° C. for 30 sec, and extension at 72° C. for 30 sec, followed by a final extension cycle of 72° C. for 5 min. The PCR products were first separated on a 1.2% agarose gel by DNA electrophoresis. The PCR products were cloned into the TOPO vector (Invitrogen, San Diego, Calif.) according to manufacturer's instructions. The clones were then sequenced using an automated DNA sequencer using standard sequencing methods.
TRANSMISSION ELECTRON MICROSCOPY (TEM), ENERGY DISPERSIVE X-RAY ANALYSIS (EDX) AND ELECTRON DIFFRACTION: The washed particles were mounted on carbon-coated copper grids. Micrographs were obtained using a Philips FEG200 operating at 200 kV. EDX spectra were obtained using a Noran Voyager system attached to the TEM. Electron diffraction for single crystals was also obtained on the Philips TEM.

### Results and Discussion

We adapted a polymerase chain reaction (PCR) method²⁵ to detect for the presence of peptide displaying phage DNA on the silver nanoparticles using a specific set of DNA primers. If some of the peptide displaying phages were resistant to acid elution and remain bound to the nanoparticles, then the presence of a phage DNA fragment after the PCR reaction indicates the presence of phage DNA. The DNA primers used for PCR amplified a 0.33 kilobase (kb) region of the phage DNA including the region encoding for the displayed peptide (see supplemental information). The silver nanoparticles are incubated in lysis buffer A (100 mM Tris-HCl, pH 8.2, 10 mM EDTA, 1% Triton X-100) and heated to 95° C. for 10 min to disrupt the phage coat resulting in phage DNA release.

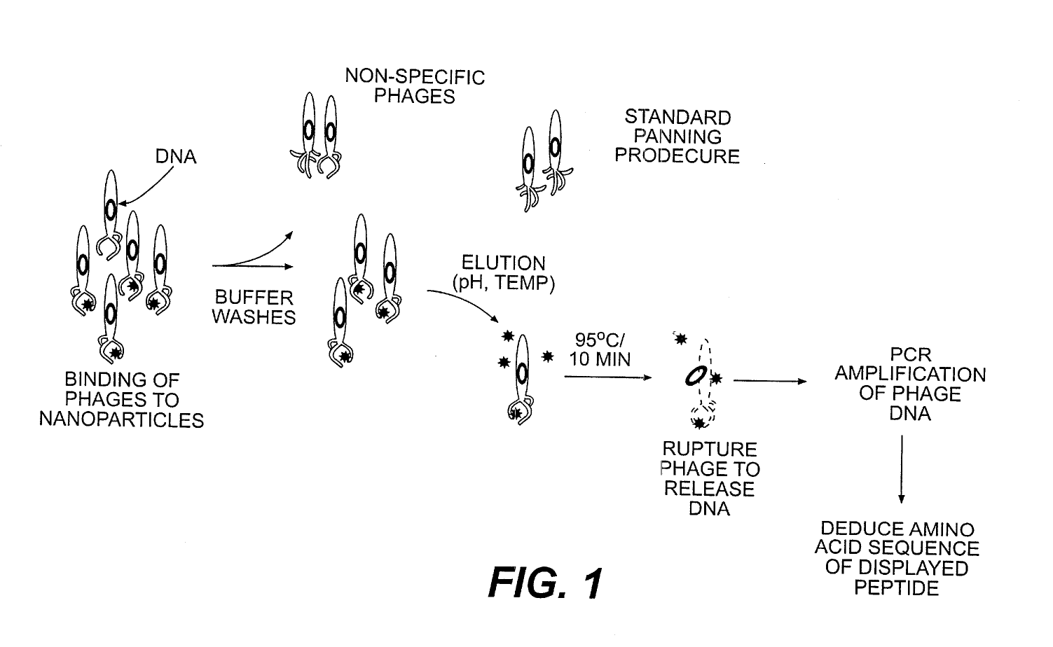

The PCR reaction mix was directly added to the silver nanoparticles and placed in a thermocycler for PCR amplification. Following the PCR reaction, the samples were separated on a 1.2% agarose gel (FIG. 1). The PCR amplification of the silver nanoparticles revealed the presence of 0.33 kb fragment, indicative of the presence of peptide displaying phages on the silver nanoparticles even after acid elution (FIG. 1, lane 1). In order to rule out phage DNA contamination, we used acid eluted zinc sulfide (ZnS) nanoparticles incubated with phage DNA as our control. In the case of ZnS nanoparticles, no DNA fragment was visible on the agarose gel (FIG. 1, lane 2). As a comparison, PCR amplification of 50 non-specific peptide-displaying phages used directly from the phage peptide library confirmed the amplification of the 0.33 kp DNA fragment (FIG. 1, lane 3). Although we can repeatedly detect 5-10 peptide displaying phages using the PCR method (supplemental figure S2), optimization of PCR conditions should allow for the detection of a single phage particle.

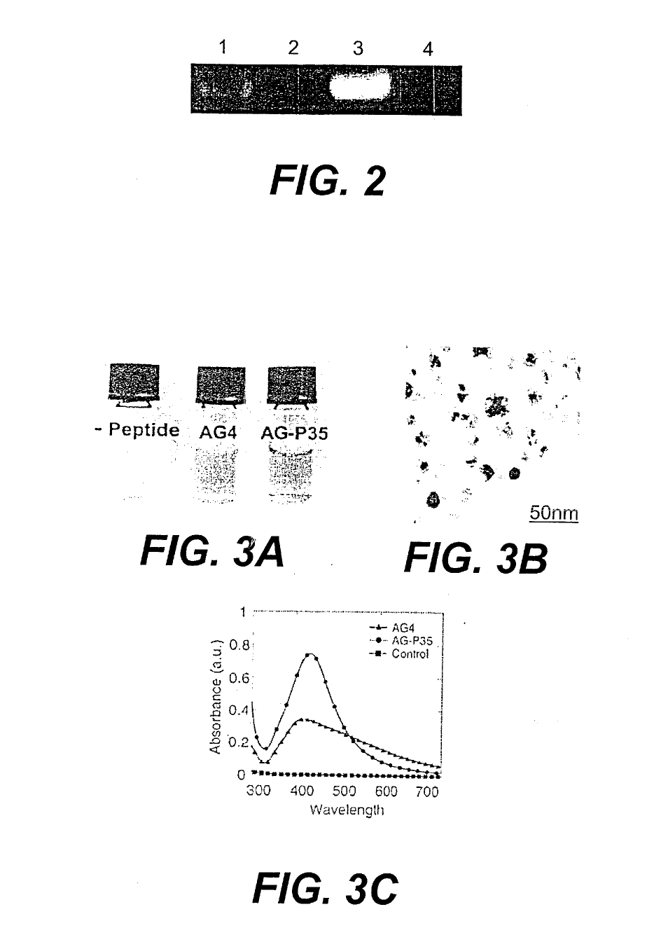

The specificity of the PCR reaction was indicated by the absence of an amplified DNA fragment when either one of the two DNA primers is omitted in the reaction (FIG. 1, lane 4). The DNA fragments were then cloned into a plasmid and DNA sequencing was acid sequences of the PCR identified silver-binding peptides are shown in Table 1. In all, 14 new silver binding sequences were identified by the PCR method. Some of the peptides identified by the PCR method were chemically synthesized and tested for their ability to reduce silver ions to metallic silver. Similar to the AG4 peptide, several peptides were capable of reducing silver ions to metallic silver. As an example, the synthesis of silver nanoparticles by AG-P35 was chosen for further analysis. As shown in FIG. 2, incubation of the AG-P35 peptide with 0.2 mM silver nitrate resulted in the formation of a yellowish-red colored solution after incubation for 24-48 hr at room temperature, similar to the AG4 peptide. Notably, the AG-P35 peptide exhibited significantly increased silver reduction activity when compared to that of AG4 peptide. The UV-Vis spectrum of AG-P35 exhibited an intense peak centered at 430 nm with a full width at half maximum (FWHM) of 120 nm, while the UV-Vis spectrum of AG4 was centered at 420 nm with a FWHM of 230 nm. Transmission electron microscopy (TEM) analysis of the AG-P35 synthesized silver nanoparticles showed the presence of small spherical silver nanoparticles with an average diameter of 52 nm±13.2 nm (FIG. 2B). In contrast, silver nanoparticles synthesized using AG4 peptide exhibited polyhedral shapes (spheres, triangles, hexagons) and were relatively larger with an average diameter of 102 nm±28 nm (supplemental figure S4) as previously described.²² The UV-Vis absorption spectrum is dependent on the size and shape of the silver nanoparticles.²⁶ The size and shape differences between the AG4 and AG-P35 synthesized silver nanoparticles was confirmed by differences in their absorption profiles. Energy dispersive X-ray (EDX) analysis confirmed that the AG-P35 synthesized nanoparticles were composed of silver (supplemental figure S3). The fact that different particle shapes were observed for different peptides supports the hypothesis that these biological templates not only nucleate, but control/direct subsequent growth of the inorganic material.

The PCR method was also used to identify peptides that remain bound to cobalt nanoparticles. However, in this case we decided to investigate in detail the utility of the PCR method as an alternative to the traditional panning approach. In order to address this issue, we compared the peptide sequences obtained before elution and after acid elution to the peptide sequences obtained after 4 rounds of regular panning. Three different sets of cobalt nanoparticles were incubated with the phage peptide display library. After several buffer washes, one set was acid eluted with 0.2 M glycine-HCl, pH 2.2 for 20 min and then used for the PCR amplification reaction (Set I), the second set was only washed several times in buffer and then subjected to PCR amplification (Set II) and the third set underwent the traditional 4 rounds of panning (Set III). The DNA agarose gel electrophoresis of the PCR reaction from both the eluted and uneluted cobalt nanoparticles revealed the presence of the 0.33 kb diagnostic phage DNA fragment (FIG. 3). It is not surprising to find peptide displaying phages bound to the non-eluted cobalt nanoparticle sample; nonetheless, one can clearly see differences in the intensity of the phage DNA fragment from uneluted and eluted cobalt nanoparticles (compare FIG. 3, lane 2 versus lane 3). The intensity of the DNA fragment by agarose gel electrophoresis provided a qualitative estimate on the number of peptide displaying phages present on the cobalt nanoparticles. Based on the relative intensities of the DNA fragments, the uneluted cobalt nanoparticles contained more peptide displaying phages bound to the surface (FIG. 3, lane 2) compared to the acid eluted nanoparticles (FIG. 3, lane 3).

The PCR fragments from both the sets were cloned and sequenced. The amino acid sequences of the cobalt binding peptides are listed in Table 2. As expected, the sequences of the uneluted nanoparticles (Set II) contained three groups of sequences, one group that was the same set of sequences as that from the eluted nanoparticles (Set I), a second set that was similar to the sequences obtained from the regular panning (Set III), and a third sequence set that was absent from either of the other two groups. One would expect that Set II sequences would represent all of the sequences contained within Set I and Set III; however, seven of the sequences did not belong to either Set I or Set III. This can be explained by the fact that a larger number of clones would have to be sequenced in order to fully sample the diversity set. But more importantly, the sequence data clearly showed that a large amino acid sequence space was being lost by the regular panning method. Over 35 independent peptide displaying phages from the regular panning method were sequenced—resulting in the selection panning method. This confirmed that the PCR method identified a unique set of peptide sequences that were not recovered by the regular panning techniques.

Based on the sequences obtained in Set II, it is possible to eliminate the labor-intensive procedures of phage amplification and directly obtain sequence information of interacting peptides in a single step using the PCR method. Analysis of both the silver and cobalt nanoparticle-binding peptides revealed some interesting sequence characteristics.

For example, all the silver binding sequences obtained by the regular panning methods lacked the amino acid cysteine (C). Cysteine is a thiol-containing amino acid that binds to silver and gold ions. Four of the sequences obtained by the PCR method contained cysteine. Furthermore, the amino acids glutamic acid (E), histidine (H), isoleucine (1), and tryptophan (W) were present in the sequences obtained by the PCR method but were absent in all of the sequences obtained by regular panning. For the cobalt binding peptides, the sequences obtained by regular panning lacked the amino acids cysteine (C), glutamic acid (E), histidine (H), isoleucine (I), tryptophan (W) and tyrosine (Y). It was surprising that these sequences lacked histidine and glutamic acid—-amino acids that are known to bind to cobalt.¹²⁻¹⁴ It is plausible that our high stringency wash conditions used in selecting the cobalt binding sequences eliminated amino acid sequences that contained these amino acids. We believe that this is unlikely since peptide sequences in Set II, obtained using the same stringency wash conditions, contained histidine residues. An alternative explanation could be that during successive rounds of panning, the histidine containing sequences were diluted out by the dominant stronger-binding sequences that were identified in the fourth round.

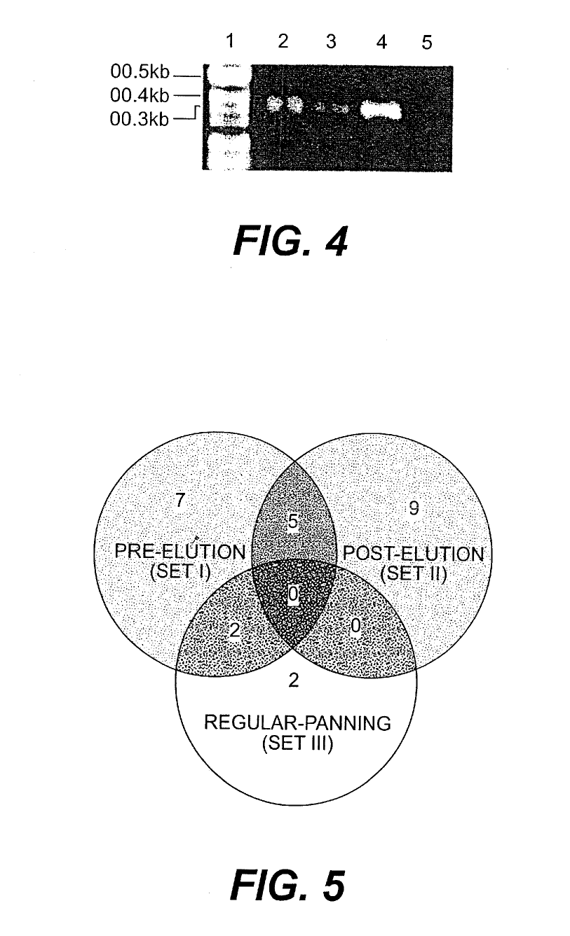

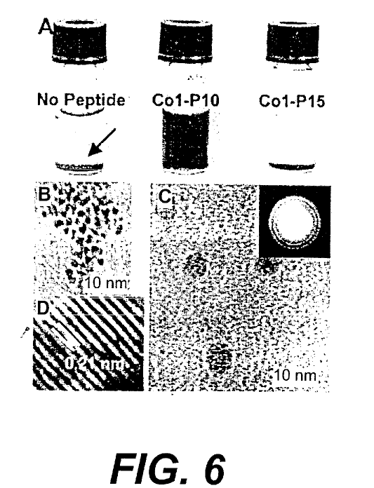

In order to address whether some of the cobalt binding sequences identified in Set I or II would function as templates in the synthesis of cobalt-platinum (CoPt) nanoparticles, the synthesis of CoPt nanoparticles was performed in the presence of the cobalt binding peptides. CoPt is a magnetic alloy and is a candidate for ultrahigh-density magnetic recording media because of its enhanced magnetic anisotropy and other properties.²⁷ Random nucleation and growth of magnetic nanoparticles results in large sizes, and broad size distributions, which adversely affect the magnetic properties. It is possible that by using biological templates that nucleate metal ions, the random nanoparticle nucleation and growth process can be more controlled. Peptide (10 mg/ml) was added to a solution containing 1 mM ammonium tetrachloroplatinate and 1 mM cobalt acetate tetrahydrate in 0.1 M HEPES buffer pH 7.5. The solutions were mixed and stored for 4 hrs to overnight at 4° C. 10 μl of 25 mM sodium borohydride was then slowly added to the solution to reduce the metallic precursors. The addition of reductant resulted in the formation of a homogenous greenish solution (FIG. 4). Prolonged standing at room temperature resulted in the precipitation of the CoPt particles in tubes containing the Co1-P15 peptide or in the absence of peptide (FIG. 4A). In contrast, the solution containing Co1-P10 or Co2-P2 peptide remained homogenous for several hours. This suggested that the stabilization of the CoPt nanoparticles was achieved by the presence of the peptides.

Similarly, Co1-P1 as well as the peptides from the regular panning method all resulted in the precipitation of the metal alloy. Analysis of the Co1-P10 stabilized CoPt solution by transmission electron microscopy (TEM) revealed the presence of discrete nanoparticles with an average diameter of 3.5 nm±0.5 nm (FIGS. 4B and 4C). High-resolution TEM (HRTEM) image showing the lattice fringes of the nanoparticles demonstrated the crystalline nature of the nanoparticles (FIG. 4D). The lattice spacing is 0.21 nm, which is close to the value of [111] facet of CoPt, which is 0.217 nm (PDF#43-1358). EDX analysis of the nanoparticles confirmed the presence of Co and Pt (supplemental figure S3).

In conclusion, we have shown here the identification of biological templates that can be used in the nucleation and growth of inorganic nanoparticles using a PCR-based approach. The PCR-based approach of screening peptide-displaying phages allows for the rapid identification of strongly interacting amino acid sequences that would be likely absent from the sequences obtained via regular phage peptide display panning methods. Albeit, with either method, screening of the binding peptides will have to be done in order to obtain the desired properties of controlled nucleation and growth.

The following references cited in the text above are incorporated by reference as if set forth in full herein.
1) Dameron, C. T., et al Nature 1989, 338, 596-597.
2) Meldrum, F. C., Mann, S., Heywood, B. R., Frankel, R. B. & Bazylinksi, D. A. Proc. R. Soc. Lond. B 1993, 251, 238-242.
3) Schueler, D.; Frankel, R. B. Appl. Microbiol. Biotechnol. 1999, 52, 464-473.
4) Klaus, T.; Joerger, R.; Olsson, E.; Granqvist, C. G. Proc. Natl. Acad. Sci. U.S.A. 1999, 96, 13611-13614.
5) Klaus-Joerger, T., Joerger, R., Olsson, E., & Granqvist, C-G. Trends Biotechnol. 2001, 19, 15-20.
6) Mann, S. Biomineralization: Principles and concepts in bioinorganic materials chemistry.; Oxford University Press: Oxford, 2001.
7) Nair, B.; Pradeep, T. Cryst. Growth Design 2002, 2, 293-298.
8) Kroger, N.; Deutzmann, R.; Sumper, M. Science 1999, 286, 1129-1132.
9) Zhou, Y., Shimizu, K. Cha, J. N., Stucky, J. D. & Morse, D. E. Angew. Chem. Int. Edn Engl. 1999, 38, 780-782.
10) Cha, J. N.; Katsuhiko, K.; Zhou, Y.; Christiansen, S. C.; Chmelka, B. F.; Stucky, G. D.; Morse, D. E. Proc. Natl. Acad. Sci. USA 1999, 96, 361-365.
11) Douglas, T.; Strable, E.; Willits, D.; Aitouchen, A.; Libera, M.; Young, M. Adv. Mater. 2002, 14, 415-418.
12) Gruen, L. C. Biochim. Biophys. Acta 1975, 386, 270-274.
13) Stillman, M. J.; Shaw, C. F.; Suzuki, K. T. Metallothioneins; VCH Publishers: New York, 1992.
14) Bar-Or, D.; Curtis, G.; Rao, N.; Bampos, N.; Lau, E. Eur. J. Biochem. 2001, 268, 42-47.
15) Slocik, J. M.; Moore, J. T.; Wright, D. W. Nano Lett. 2002, 2, 169-172.
16) Brown, S. Nature Biotechnol. 1997, 15, 269-272.
17) Brown, S.; Sarikaya, M.; Johnson, E. J. Mol. Biol. 2000, 299, 725-735.
18) Gaskin, D. J. H.; Starck, K.; Vulfson, E. N. Biotech. Letts. 2000, 22, 1211-1216.
19) Whaley, S. R., English, D. S., Hu, E. L., Barbara, P. F. & Belcher, A. M. Nature 2000, 405, 665-668.
20) Lee, S.-W.; Mao, C.; Flynn, C. E.; Belcher, A. M. Science 2002, 296, 892-895. 95-100.
22) Naik, R. R.; Stringer, S. J.; Agarwal, G. A.; Jones, S. E.; Stone, M. O. Nature Maters. 2002, 1, 169-172.
23) Wang, S.; Humphreys, E. S.; Chung, S.-Y.; Delduco, D. F.; Lustig, S. R.; Wang, H.; Parker, K. N.; Rizzo, N. W.; Subramoney, S.; Chiang, Y.-M.; Jagota, A. Nature Maters. 2003, 2, 196-200.
24) Rodi, D. J., Makowski, L. Anal. Biotechnol. 1999, 10, 87-93.
25) Estell, D. A.; Murray, C. J.; Tijerina, P.; Chen, Y. Patent 01/79479 A2, 2001.
26) Schatz, G. C.; Van Duyne, R. P. Handbook of vibrational spectroscopy.; Wiley: New York, 2002.
27) Hyeon, T. Chem. Comm. 2003, 1, 927-934.

TABLE 1
Silver-binding peptides.

| | |
|---|---|
| PCR Method | Regular panning |

| | | | |
|---|---|---|---|
| AG-P1 | KFLQFVCLCVCP (8) | AG3 | AYSSGAPPMPPP (22) |
| AG-P2 | AVLMQKYHQLGP (9) | AG4 | NPSSLFRYLPSD (23) |
| AG-P3 | IRPAIHIIPISH (10) | AG5 | SLATQPPRTPPV (24) |
| AG-P4 | NVIRASPPDTSY (11) | | |
| AG-P5 | LAMPNTQADAPF (12) | | |
| AG-P6 | QQNVPASGTCSI (13) | | |
| AG-P10 | NAMPGMVAWLCR (14) | | |
| AG-P11 | HNTSPSPIILTP (15) | | |
| AG-P12 | ASQTLLLPVPPL (16) | | |
| AG-P14 | YNKDRYEMQAPP (17) | | |
| AG-P18 | TLLLLAFVHTRH (18) | | |
| AG-P27 | PWATAVSGCFAP (19) | | |
| AG-P28 | SPLLYATTSNQS (20) | | |
| AG-P35 | WSWRSPTPHVVT (21) | | |

(SEQ ID NOS. IN PARENTHESIS)

TABLE 2
Cobalt-binding peptides.

| | | |
|---|---|---|
| Eluted cobalt nanoparticles | Uneluted cobalt nanoparticles | Regular panning |
| (Set I) | (Set II) | (Set III) |

| | | | | | |
|---|---|---|---|---|---|
| Co1-P1 | HSVRWLLPGAHP (34) | Co2-P4 | HSVRWLLPGAHP (34) | Co3-P12 | GTSTFNSVPVRD (50) |
| Co1-P2 | HETNPPATIMPH (35) | Co2-P3 | SAPNLNALSAAS (44) | Co3-P13 | SAPNLNALSAAS (44) |
| Co1-P3 | WASAAWLVHSTI (36) | Co2-P5 | SVSVGMKPSPRP (45) | Co3-P1 | SVSVGMKPSPRP (45) |
| Co1-P4 | SPLQVLPYQGYV (37) | Co2-P8 | SPLQVLPYQGYV (37) | Co3-P16 | VPTNVQLQTPRS (32) |
| Co1-P5 | ESIPALAGLSDK (38) | Co2-P1 | SLTQTVTPWAFY (46) | | |
| Co1-P6 | GVLNAAQTWALS (25) | Co2-P7 | TNLDDSYPLHHL (47) | | |
| Co1-P8 | TPNSDALLTPAL (39) | Co2-P6 | TPNSDALLTPAL (39) | | |
| Co1-P10 | HYPTLPLGSSTY (26) | Co2-P12 | HYPTLPLGSSTY (26) | | |
| Co1-P13 | HAMRPQVHPNYA (40) | Co2-P9 | TQQTDSRPPVLL (29) | | |
| Co1-P15 | QYKHHPQKAAHI (27) | Co2-P14 | QYKHHPQKAANI (27) | | |
| Co1-P16 | YGNQTPYWYPHR (41) | Co2-P11 | TFPSHLATSTQP (30) | | |
| Co1-P17 | HPPTDGMVPSPP (28) | Co2-P13 | QNFLQVIRNAPR (48) | | |
| Co1-P18 | TWQPFGMRPSDP (42) | Co2-P2 | KLHSSPHTPLVQ (49) | | |
| Co1-P21 | TGDVSNNPNVTL (43) | Co2-P17 | QLLPLTPSLLQA (31) | | |

TABLE 3
Peptides used in this study.

| | | |
|---|---|---|
| | Isoelectric | Panning |
| Amino Acid sequnce | pH (pl) | method |

Silver binding peptides

| | | | |
|---|---|---|---|
| AG-P3 | IRPAIHIPPISH (10) | 9.7 | PCR |
| AG-P4 | NVIRASPPDTSY (11) | 5.8 | PCR |
| AG-P5 | LAMPNTQADAPF (12) | 3.8 | PCR |
| AG-P28 | SPLLYATTSNQS (20) | 5.2 | PCR |
| AC-P35 | WSWRSPTPHVVT (21) | 9.7 | PCR |
| AG-4 | NPSSLFRYLPSD (23) | 6.1 | Regular |

Cobalt binding peptides

| | | | |
|---|---|---|---|
| Co1-Pa | HSVRWLLPGAHP (34) | 9.7 | PCR |
| Co2-P2 | KLHSSPHTLPVQ (49) | 8.6 | KR |
| Co1-P10 | HYPTLPLGSSTT (26) | 6.7 | PCR |
| Co1-P15 | QYKHHPQKAAHI (27) | 9.7 | PCR |

(SEQ ID NOS. IN PARENTHESIS)

What is claimed is:

1. A method for identifying peptides which can bind to an inorganic material using a combinatorial phage display library comprising:
a. incubating a combinatorial phage peptide display library with a target inorganic material for a time sufficient so that the inorganic material will bind to peptides expressed by the phage of the library;
b. eluting the library so as to collect the phage bound to the target inorganic material;
c. rupturing the phage so as to release the nucleic acid of the phage bound to the target inorganic material;
d. amplifying the nucleic acid of the phage bound to the target inorganic material using a polymerase-chain reaction (PCR); and
e. sequencing said nucleic acid so as to determine the sequence of the peptides coded by the nucleic acid of the phage which can bind to the target inorganic material.

2. The method of claim 1 further comprising a step of washing the phages with a buffer solution so as to remove phages that do not specifically bind to the inorganic material;

3. The method of claim 1 further comprising a step of expressing the peptide identified by the method of claim 1.

4. The method of claim 1 wherein steps a-b are repeated so as to increase the purification of the phage bound to the inorganic material.

5. The method of claim 1 wherein the peptide expressed by the phage is capable of catalyzing the deposition or precipitation, or controlling or directing the growth of the target inorganic material.

6. The method of claim 1 wherein the inorganic material is selected from the group consisting of silver, gold, platinum, cobalt, silica, iron, zinc, tin, palladium, gadolinium, germanium, aluminum, antimony, beryllium, cadmium, copper, lead, selenium, cobalt platinum and oxides thereof, ruby and Na+montmorillonite.

7. The method of claim 1 wherein the inorganic material is a radioactive material.

8. The method of claim 7 wherein the radioactive material is selected from the group consisting of radioactive cobalt and uranium.

9. A peptide identified by the method of claim 1.

10. A peptide according to claim 9 wherein said peptide binds to a material selected from the group consisting of silver, gold, platinum, cobalt, silica, iron, zinc, tin, palladium, gadolinium, germanium, aluminum, antimony, beryllium, cadmium, copper, lead, selenium and oxides thereof, ruby and Na⁺ montmorillonite.

11. A peptide according to claim 9 wherein said peptide binds to silica, and wherein the sequence of said peptide is SEQ ID NO: 1.

12. A peptide according to claim 9 wherein said peptide binds to silver, and wherein the sequence of said peptide is selected from the group consisting of SEQ ID NOS: 8-21.

13. A peptide according to claim 9 wherein said peptide binds to cobalt oxide, and wherein the sequence of said peptide is selected from the group consisting of SEQ ID NOS: 25-33.

14. A peptide according to claim 9 wherein said peptide binds to iron oxide, and wherein the sequence of said peptide is selected from the group consisting of SEQ ID NOS: 51-56.

15. A peptide according to claim 9 wherein said peptide binds to germanium oxide, and wherein the sequence of said peptide is selected from the group consisting of SEQ ID NOS: 57-60.

16. A peptide according to claim 9 wherein said peptide binds to tin oxide, and wherein the sequence of said peptide is selected from the group consisting of SEQ ID NOS: 61-64.

17. A peptide according to claim 9 wherein said peptide binds to titanium oxide, and wherein the sequence of said peptide is selected from the group consisting of SEQ ID NOS: 65-68.

18. A peptide according to claim 9 wherein said peptide binds to gadolinium, and wherein the sequence of said peptide is selected from the group consisting of SEQ ID NOS: 69-75.

19. A peptide according to claim 9 wherein said peptide binds to ruby, and wherein the sequence of said peptide is selected from the group consisting of SEQ ID NOS: 76-78.

20. A peptide according to claim 9 wherein said peptide binds to cobalt platinum, and wherein the sequence of said peptide is selected from the group consisting of SEQ ID NOS: 83-86.

21. A peptide according to claim 9 wherein said peptide binds to palladium, and wherein the sequence of said peptide is selected from the group consisting of SEQ ID NOS: 87-89.

22. A peptide according to claim 9 wherein said peptide binds to zinc oxide, and wherein the sequence of said peptide is selected from the group consisting of SEQ ID NOS: 90-92.

23. A peptide according to claim 9 wherein said peptide binds to gold, and wherein the sequence of said peptide is SEQ ID NO: 93.

24. A peptide according to claim 9 wherein said peptide binds to Na+montmorillonite, and wherein the sequence of said peptide is selected from the group consisting of SEQ ID NOS: 94-95.

25. A method for obtaining phage which can express a peptide which can bind to an inorganic material using a combinatorial phage display library comprising:
a. incubating a combinatorial phage display peptide library with a target inorganic material which will bind to peptides expressed by the phage of the library;
b. eluting the library so as to collect the phage bound to the target inorganic material; and
c. amplifying the nucleic acid of the phage bound to the target inorganic material using a polymerase-chain reaction (PCR) and sequencing said nucleic acid so as to determine the sequence of the peptides coded by the nucleic acid of the phage which can bind to the target inorganic material.

26. A method of initiating the deposition or precipitation of an inorganic material on a nanometric scale comprising expressing a peptide obtained by the method of claim 1, and using said peptide as a template to initiate the deposition or precipitation of said inorganic material.

27. The method according to claim 26 wherein said inorganic material is selected from the group consisting of silver, gold, platinum, cobalt, silica, iron, zinc, tin, palladium, gadolinium, germanium, and oxides thereof.

28. A nucleic acid encoding a peptide according to claim 9.

29. A nucleic acid according to claim 28 wherein the nucleic acid encodes a peptide having a sequence selected from the group consisting of SEQ ID NOS: 1, 8-21, 25-33, 51-78, and 83-95.

30. A method for recovering an inorganic material using a peptide according to claim 9 comprising:
a. providing the peptide of claim 9 in an amount effective to reduce or eliminate the inorganic ingredient to which said peptide will bind;
b. introducing said peptide into a solution containing the inorganic material to be removed and maintaining the peptide in said solution for a time sufficient for the peptide to bind with said inorganic material; and
c. removing said peptide after it has become bound to said inorganic material so as to recover the inorganic material.

31. A method for identifying peptides which can bind to a stable inorganic element or a stable inorganic complex of said element using a combinatorial phage display library comprising:
a. incubating a combinatorial phage display peptide library with a target inorganic element or complex which will bind to peptides expressed by the phage of the library;
b. eluting the library so as to collect the phage bound to the target inorganic element or complex;
c. isolating the nucleic acid of the phage bound to the target inorganic element or complex;
d. amplifying the nucleic acid of the phage bound to the target inorganic element or complex using a polymerase-chain reaction (PCR); and
e. sequencing said nucleic acid so as to determine the sequence of the peptides coded by the nucleic acid of the phage which can bind to the target inorganic element or complex.

32. A method for identifying peptides which can bind to a carbon nanotube using a combinatorial phage display library comprising:
a. incubating a combinatorial phage peptide display library with a carbon nanotube for a time sufficient so that the carbon nanotube will bind to peptides expressed by the phage of the library;
b. eluting the library so as to collect the phage bound to the carbon nanotube;
c. rupturing the phage so as to release the nucleic acid of the phage bound to the carbon nanotube;
d. amplifying the nucleic acid of the phage bound to the carbon nanotube using a polymerase-chain reaction (PCR); and
e. sequencing said nucleic acid so as to determine the sequence of the peptides coded by the nucleic acid of the phage which can bind to the carbon nanotube.

33. The method of claim 32 further comprising a step of expressing the peptide identified by the method of claim 32.

34. A peptide identified by the method of claim 33.

35. A peptide according to claim 34 wherein the sequence of said peptide is selected from the group consisting of SEQ ID NOS: 79-82.

36. A nucleic acid encoding a peptide according to claim 35.

37. A silver binding peptide having an amino acid sequence selected from the group consisting of SEQ ID NOS: 8-24.

38. A peptide capable of binding a noble metal selected from the group consisting of SEQ ID NOS: 8-24, 83-89 and 93.


SEQUENCE LISTING (97 sequences)

SEQ ID NO: 1 - protein, 12 aa, Escherichia coli
KPHHHHTHHMYT

SEQ ID NO: 2 - protein, 12 aa, Escherichia coli
LPHHHHLHTKLP

SEQ ID NO: 3 - protein, 12 aa, Escherichia coli
KPSHHHHHTGAN

SEQ ID NO: 4 - protein, 11 aa, Escherichia coli
APGHHHWHIHH

SEQ ID NO: 5 - protein, 12 aa, Escherichia coli
MSPHPHPRHHHT

SEQ ID NO: 6 - protein, 12 aa, Escherichia coli
MSPHHMHHSHGH

SEQ ID NO: 7 - protein, 12 aa, Escherichia coli
MSASSYASFSWS

SEQ ID NO: 8 - protein, 12 aa, Escherichia coli
KFLQFVCLGVGP

SEQ ID NO: 9 - protein, 12 aa, Escherichia coli
AVLMQKYHQLGP

SEQ ID NO: 10 - protein, 12 aa, Escherichia coli
IRPAIHIIPISH

SEQ ID NO: 11 - protein, 12 aa, Escherichia coli
NVIRASPPDTSY

SEQ ID NO: 12 - protein, 12 aa, Escherichia coli
LAMPNTQADAPF

SEQ ID NO: 13 - protein, 12 aa, Escherichia coli
QQNVPASGTCSI

SEQ ID NO: 14 - protein, 12 aa, Escherichia coli
NAMPGMVAWLCR

SEQ ID NO: 15 - protein, 12 aa, Escherichia coli
HNTSPSPIILTP

SEQ ID NO: 16 - protein, 12 aa, Escherichia coli
ASQTLLLPVPPL

SEQ ID NO: 17 - protein, 12 aa, Escherichia coli
YNKDRYEMQAPP

SEQ ID NO: 18 - protein, 12 aa, Escherichia coli
TLLLLAFVHTRH

SEQ ID NO: 19 - protein, 12 aa, Escherichia coli
PWATAVSGCFAP

SEQ ID NO: 20 - protein, 11 aa, Escherichia coli
SPLYATTSNQS

SEQ ID NO: 21 - protein, 12 aa, Escherichia coli
WSWRSPTPHVVT

SEQ ID NO: 22 - protein, 12 aa, Escherichia coli
AYSSGAPPMPPF

SEQ ID NO: 23 - protein, 12 aa, Escherichia coli
NPSSLFRYLPSD

SEQ ID NO: 24 - protein, 12 aa, Escherichia coli
SLATQPPRTPPV

SEQ ID NO: 25 - protein, 12 aa, Escherichia coli
GVLNAAQTWALS

SEQ ID NO: 26 - protein, 12 aa, Escherichia coli
HYPTLPLGSSTY

SEQ ID NO: 27 - protein, 12 aa, Escherichia coli
QYKHHPQKAAHI

SEQ ID NO: 28 - protein, 12 aa, Escherichia coli
HPPTDGMVPSPP

SEQ ID NO: 29 - protein, 12 aa, Escherichia coli
TQQTDSRPPVLL

SEQ ID NO: 30 - protein, 12 aa, Escherichia coli
TFPSHLATSTQP

SEQ ID NO: 31 - protein, 12 aa, Escherichia coli
QLLPLTPSLLQA

SEQ ID NO: 32 - protein, 12 aa, Escherichia coli
VPTNVQLQTPRS

SEQ ID NO: 33 - protein, 12 aa, Escherichia coli
CFSQLNALPLIL

SEQ ID NO: 34 - protein, 12 aa, Escherichia coli
HSVRWLLPGAHP

SEQ ID NO: 35 - protein, 12 aa, Escherichia coli
HETNPPATIMPH

SEQ ID NO: 36 - protein, 12 aa, Escherichia coli
WASAAWLVHSTI

SEQ ID NO: 37 - protein, 12 aa, Escherichia coli
SPLQVLPYQGYV

SEQ ID NO: 38 - protein, 12 aa, Escherichia coli
ESIPALAGLSDK

SEQ ID NO: 39 - protein, 12 aa, Escherichia coli
TPNSDALLTPAL

SEQ ID NO: 40 - protein, 12 aa, Escherichia coli
HAMRPQVHPNYA

SEQ ID NO: 41 - protein, 12 aa, Escherichia coli
YGNQTPYWYPHR

SEQ ID NO: 42 - protein, 12 aa, Escherichia coli
TWQPFGMRPSDP

SEQ ID NO: 43 - protein, 12 aa, Escherichia coli
TGDVSNNPNVTL

SEQ ID NO: 44 - protein, 12 aa, Escherichia coli
SAPNLNALSAAS

SEQ ID NO: 45 - protein, 12 aa, Escherichia coli
SVSVGMKPSPRP

SEQ ID NO: 46 - protein, 12 aa, Escherichia coli
SLTQTVTPWAFY

SEQ ID NO: 47 - protein, 12 aa, Escherichia coli
TNLDPSYPLHHL

SEQ ID NO: 48 - protein, 12 aa, Escherichia coli
QNFLQVIRNAPR

SEQ ID NO: 49 - protein, 12 aa, Escherichia coli
KLHSSPHTPLVQ

SEQ ID NO: 50 - protein, 12 aa, Escherichia coli
GTSTFNSVPVRD

SEQ ID NO: 51 - protein, 12 aa, Escherichia coli
LPDSHHYKSDDH

SEQ ID NO: 52 - protein, 12 aa, Escherichia coli
QHMQQPQTQGIQ

SEQ ID NO: 53 - protein, 12 aa, Escherichia coli
SLYSNPTVPYSY

SEQ ID NO: 54 - protein, 12 aa, Escherichia coli
LPGSHQYQQQLL

SEQ ID NO: 55 - protein, 12 aa, Escherichia coli
QHITQSIWPGVR

SEQ ID NO: 56 - protein, 12 aa, Escherichia coli
QQLPKNGCLPAV

SEQ ID NO: 57 - protein, 12 aa, Escherichia coli
SLKMPHWPHLLP

SEQ ID NO: 58 - protein, 12 aa, Escherichia coli
TGHQSPGAYAAH

SEQ ID NO: 59 - protein, 12 aa, Escherichia coli
SFLYSYTGPRPL

SEQ ID NO: 60 - protein, 12 aa, Escherichia coli
HATGTHGLSLSH

SEQ ID NO: 61 - protein, 12 aa, Escherichia coli
KNAGQYPPSALM

SEQ ID NO: 62 - protein, 12 aa, Escherichia coli
SPSHSADHTPPT

SEQ ID NO: 63 - protein, 12 aa, Escherichia coli
TPTLRSMSSLLF

SEQ ID NO: 64 - protein, 12 aa, Escherichia coli
STLTQSTSSLVA

SEQ ID NO: 65 - protein, 12 aa, Escherichia coli
QPYLFATDSLIK

SEQ ID NO: 66 - protein, 12 aa, Escherichia coli
DLNYFTLSSKRE

SEQ ID NO: 67 - protein, 12 aa, Escherichia coli
SSWSSPITTAAV

SEQ ID NO: 68 - protein, 12 aa, Escherichia coli
GHTHYHAVRTQT

SEQ ID NO: 69 - protein, 12 aa, Escherichia coli
TTFSHYANQVHR

SEQ ID NO: 70 - protein, 12 aa, Escherichia coli
AETVESCLAKSH

SEQ ID NO: 71 - protein, 12 aa, Escherichia coli
LPYGTSNRHAPV

SEQ ID NO: 72 - protein, 12 aa, Escherichia coli
SLASYLQSWLGS

SEQ ID NO: 73 - protein, 12 aa, Escherichia coli
TKNMLSLPVGPG

SEQ ID NO: 74 - protein, 12 aa, Escherichia coli
EDNLAVRSQRIM

SEQ ID NO: 75 - protein, 12 aa, Escherichia coli
HAFQGLPLPSFT

SEQ ID NO: 76 - protein, 12 aa, Escherichia coli
AHRPLSANPFTA

SEQ ID NO: 77 - protein, 12 aa, Escherichia coli
HHKPWHPGKLLI

SEQ ID NO: 78 - protein, 12 aa, Escherichia coli
HSNWRVPSPWQL

SEQ ID NO: 79 - protein, 12 aa, Escherichia coli
HSSYWYAFNNKT

SEQ ID NO: 80 - protein, 12 aa, Escherichia coli
HTSYWYAFNTKT

SEQ ID NO: 81 - protein, 12 aa, Escherichia coli
YTTHVLPFAPSS

SEQ ID NO: 82 - protein, 12 aa, Escherichia coli
HAWVDWIRPIHS

SEQ ID NO: 83 - protein, 12 aa, Escherichia coli
KYHNLHSHPLHK

SEQ ID NO: 84 - protein, 12 aa, Escherichia coli
KTHSLHSPLSHK

SEQ ID NO: 85 - protein, 12 aa, Escherichia coli
HLKHLPHTLPHK

SEQ ID NO: 86 - protein, 12 aa, Escherichia coli
KLHSSPHTPLVQ

SEQ ID NO: 87 - protein, 12 aa, Escherichia coli
NFMSLPRLGHMH

SEQ ID NO: 88 - protein, 12 aa, Escherichia coli
TSNAVHPTLRHL

SEQ ID NO: 89 - protein, 12 aa, Escherichia coli
TTTKSITLTLSV

SEQ ID NO: 90 - protein, 12 aa, Escherichia coli
GLHIPTGSYSHR

SEQ ID NO: 91 - protein, 12 aa, Escherichia coli
NLLTSNSHWPPR

SEQ ID NO: 92 - protein, 12 aa, Escherichia coli
TPSATMQTRPGL

SEQ ID NO: 93 - protein, 13 aa, Escherichia coli
AYSSGAFPPMPPF

SEQ ID NO: 94 - protein, 12 aa, Escherichia coli
WPSSYLSPIPYS

SEQ ID NO: 95 - protein, 12 aa, Escherichia coli
AVTTLTLVPAGT

SEQ ID NO: 96 - DNA, 21 bp, Bacteriophage 12826
cctcgaaagcaagctgataac

SEQ ID NO: 97 - DNA, 21 bp, Bacteriophage 12826
gtaccgtaacactgagtttcg